SEQUENCE LISTING (27 sequences)

SEQ ID NO: 1 - DNA, 667 bp, Artificial Sequence
  Description of Artificial Sequence ColE1-related origin of replication (ORI) from pBluescript plasmid (residues 1158-1825)
tcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 2 - DNA, 553 bp, Artificial Sequence
  Description of Artificial Sequence DNA encoding RNAII from ColE1-related origin of replication (ORI) from pBluescript plasmid
gcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 3 - DNA, 599 bp, Artificial Sequence
  Description of Artificial Sequence DNA encoding origin of replication (ori5′) mutant 3.1 (ori 3.1), a high-copy variant of ColE1-type plasmid
ggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 4 - DNA, 663 bp, Artificial Sequence
  Description of Artificial Sequence DNA encoding origin of replication (ori5′) mutant 3.2 (ori 3.2), a high-copy variant of ColE1-type plasmid
tcttgagatcctttttttctgcgcgtaatctgctgcttgcaaacaaaaaaaccaccccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 5 - DNA, 664 bp, Artificial Sequence
  Description of Artificial Sequence DNA encoding origin of replication (ori5′) mutant 3.3 (ori 3.3), a high-copy variant of ColE1-type plasmid
tcttgagatcctttttttctgcgcgtaatctgctgcttgcaaacaaaaaaaccaccgctaccagcggtggtttgtttgccggatcaagagctaccaactctttttccgaaggtaactggcttcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 6 - DNA, 656 bp, Artificial Sequence
  Description of Artificial Sequence DNA encoding origin of replication (ori5′) mutant 3.4 (ori 3.4), a high-copy variant of ColE1-type plasmid
tcttgagatcctttttttctgcgcgtaatctgctgcttgaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 7 - DNA, 661 bp, Artificial Sequence
  Description of Artificial Sequence DNA encoding origin of replication (ori5′) mutant 2.1 (ori 2.1), a high-copy variant of ColE1-type plasmid
tcttgagatcctttttttctgcgcgtaatctgctgcttgcaaacaaaaaaaccaccggcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 8 - DNA, 664 bp, Artificial Sequence
  Description of Artificial Sequence DNA encoding origin of replication (ori5′) mutant 2.2 (ori 2.2), a high-copy variant of ColE1-type plasmid
tcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactcagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggntcctggncntttgctggccttttgctcacatgttctttcctgcgttat

SEQ ID NO: 9 - DNA, 660 bp, Artificial Sequence
  Description of Artificial Sequence DNA encoding origin of replication (ori5′) mutant 2.3 (ori 2.3), a high-copy variant of ColE1-type plasmid
tcttgagatcctttttttctgcgcgtaatctgctgcttgcaaacaaaaaaaccaccgctaccagcggtggtttgtttgccggatcaagagctaccaactctttttccgaaggtaactggcttcagcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 10 - DNA, 665 bp, Artificial Sequence
  Description of Artificial Sequence DNA encoding origin of replication (ori5′) mutant 4.1 (ori 4.1), a high-copy variant of ColE1-type plasmid
tcttgagatcctttttttctgcgcgtaatctgctgcttgcaaacaaaaaaaccaccggtgaccggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcanggtcggaacaggagagcgcacganggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 11 - DNA, 657 bp, Artificial Sequence
  Description of Artificial Sequence DNA encoding origin of replication (ori5′) mutant 5.1 (ori 5.1), a high-copy variant of ColE1-type plasmid
tcttgagatcctttttttctgcgcgtaatctgctgcttgcaaacaaaaaaaccaccgctaccagcggtggtttgtttgccggatcaagagctttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggncttttngctcacatgttctttcctgcgttat

SEQ ID NO: 12 - DNA, 662 bp, Artificial Sequence
  Description of Artificial Sequence DNA encoding origin of replication (ori5′) mutant 5.2 (ori 5.2), a high-copy variant of ColE1-type plasmid
tcttgagatcctttttttctgcgcgtaatctgctgcttgcaaacaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 13 - DNA, 660 bp, Artificial Sequence
  Description of Artificial Sequence DNA encoding origin of replication (ori5′) mutant 5.3 (ori 5.3), a high-copy variant of ColE1-type plasmid
tcttgagatcctttttttctgcgcgtaatctgctgcttgcaaacaaaaaaaccaccgcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttat

SEQ ID NO: 14 - DNA, 270 bp, Artificial Sequence
  Description of Artificial Sequence DNA encoding origin of replication (ori5′) mutant pBS, a high-copy variant of ColE1-type plasmid
tcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 15 - DNA, 264 bp, Artificial Sequence
  Description of Artificial Sequence DNA encoding origin of replication (ori5′) mutant 2.1 (ori 2.1), a high-copy variant of ColE1-type plasmid
tcttgagatcctttttttctgcgcgtaatctgctgcttgcaaacaaaaaaaccaccggcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 16 - DNA, 267 bp, Artificial Sequence
  Description of Artificial Sequence DNA encoding origin of replication (ori5′) mutant 2.2 (ori 2.2), a high-copy variant of ColE1-type plasmid
tcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactcagcaccgcctacatacctcgctctgctaatcctgttaccagtggctgctgccagtggcgataagtcgtgtcttacc

SEQ ID NO: 17 - DNA, 263 bp, Artificial Sequence
  Description of Artificial Sequence DNA encoding origin of replication (ori5′) mutant 2.3 (ori 2.3), a high-copy variant of ColE1-type plasmid
tcttgagatcctttttttctgcgcgtaatctgctgcttgcaaacaaaaaaaccaccgctaccagcggtggtttgtttgccggatcaagagctaccaactctttttccgaaggtaactggcttcagcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 18 - DNA, 266 bp, Artificial Sequence
  Description of Artificial Sequence DNA encoding origin of replication (ori5′) mutant 3.2 (ori 3.2), a high-copy variant of ColE1-type plasmid
tcttgagatcctttttttctgcgcgtaatctgctgcttgcaaacaaaaaaaccaccccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 19 - DNA, 267 bp, Artificial Sequence
  Description of Artificial Sequence DNA encoding origin of replication (ori5′) mutant 3.3 (ori 3.3), a high-copy variant of ColE1-type plasmid
tcttgagatcctttttttctgcgcgtaatctgctgcttgcaaacaaaaaaaccaccgctaccagcggtggtttgtttgccggatcaagagctaccaactctttttccgaaggtaactggcttcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 20 - DNA, 259 bp, Artificial Sequence
  Description of Artificial Sequence DNA encoding origin of replication (ori5′) mutant 3.4 (ori 3.4), a high-copy variant of ColE1-type plasmid
tcttgagatcctttttttctgcgcgtaatctgctgcttgaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 21 - DNA, 268 bp, Artificial Sequence
  Description of Artificial Sequence DNA encoding origin of replication (ori5′) mutant 4.1 (ori 4.1), a high-copy variant of ColE1-type plasmid
tcttgagatcctttttttctgcgcgtaatctgctgcttgcaaacaaaaaaaccaccggtgaccggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 22 - DNA, 259 bp, Artificial Sequence
  Description of Artificial Sequence DNA encoding origin of replication (ori5′) mutant 5.1 (ori 5.1), a high-copy variant of ColE1-type plasmid
tcttgagatcctttttttctgcgcgtaatctgctgcttgcaaacaaaaaaaccaccgctaccagcggtggtttgtttgccggatcaagagctttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 23 - DNA, 265 bp, Artificial Sequence
  Description of Artificial Sequence DNA encoding origin of replication (ori5′) mutant 5.2 (ori 5.2), a high-copy variant of ColE1-type plasmid
tcttgagatcctttttttctgcgcgtaatctgctgcttgcaaacaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 24 - DNA, 263 bp, Artificial Sequence
  Description of Artificial Sequence DNA encoding origin of replication (ori5′) mutant 5.3 (ori 5.3), a high-copy variant of ColE1-type plasmid
tcttgagatcctttttttctgcgcgtaatctgctgcttgcaaacaaaaaaaccaccgcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacc

SEQ ID NO: 25 - RNA, 112 nt, Artificial Sequence
  Description of Artificial Sequence RNAII region of Bluescript plasmid
gcaaacaaaaaaaccaccgcuaccagcggugguuuguuugccggaucaagagcuaccaacucuuuuuccgaagguaacuggcuucagcagagcgcagauaccaaauacuguu

SEQ ID NO: 26 - DNA, 10 bp, Artificial Sequence
  Description of Artificial Sequence first ten nucleotides of SEQ ID NO3
ggtttgtttg

SEQ ID NO: 27 - DNA, 11 bp, Artificial Sequence
  Description of Artificial Sequence 11 nucleotide deletion from positions 40-50 of origin mutant 3.4 (ori 3.4)
caaacaaaaaa

(19) United States
(12) Patent Application Publication
(10) Pub. No.: US 2004/0235121 A1
(43) Pub. Date: Nov. 25, 2004

(54) High copy number plasmids and their derivatives

(75) Inventor: Vaughn Smider, Alameda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Integrigen, Inc., Novato, CA

(21) Appl. No.: 10/651,654

(22) Filed: Aug. 29, 2003

Related U.S. Application Data
(60) Provisional application No. 60/407,053, filed on Aug. 29, 2002.

(51) Int. Cl. C12P 19/34; C12N 15/74

(52) U.S. Cl. 435/91.2; 435/320.1

(57) ABSTRACT

This invention provides origins of replication capable of amplifying nucleic acid at an increased copy number within a cell. In particular, the invention provides origins of replication that amplify plasmid to an increased copy number within a bacterium.

### CROSS-REFERENCES TO RELATED APPLICATIONS

This application claims the benefit of U.S. provisional application No. 60/407,053, filed Aug. 29, 2002, which is incorporated by reference herein.

### BACKGROUND OF THE INVENTION

Plasmids are commonly used as vectors for the cloning and expression of foreign genes in bacteria. It is particularly desirable, for this purpose, to use plasmids that are present in high copy number, either in order to obtain the foreign DNA in a large quantity, or in order to increase the amount of expressed product.

The production of large quantities of proteins for use as therapeutics, additives, and other myriad applications remains a challenge. Large-scale fermentation is a commonly used method, but is expensive and difficult to maintain the required quality and consistency of product. When producing proteins in bacteria, vectors that have a high copy number are generally sought because the amount of protein is often directly proportional to gene dosage.

DNA vaccination, or DNA-mediated immunization, refers to the direct introduction into a living species of plasmid or non-plasmid DNA or RNA that can cause expression of antigenic protein(s) or peptide(s) in the newly transfected cells. The nucleic acid may be introduced into tissues of the host species by variety of techniques, e.g., needle injection, particle bombardment or orally using various DNA formulations, which may be either “naked” DNA, coated microparticles, or liposomes or biodegradable microcapsules or microspheres.

Runaway replication plasmid vectors have been developed for expression of genes in bacteria. While these runaway-replication plasmid vectors have been used to produce a variety of proteins, including hGCSF and somatotropin, the amount of protein produced has been limited by such factors as the copy number, and cell death resulting from runaway replication, thus preventing the use of continuous fermentation techniques. Thus, there is a need for expression vector systems without these limitations.

Plasmids are extrachromosomal circular DNA molecules that are transferable from one bacterium to another and replicate independently of the bacterial chromosome. A given plasmid can be present in a high copy number inside a bacterial cell. The copy number is a genetic characteristic of each plasmid. For example, in the ColE1-type plasmids (such as plasmids of the families pBR, pUC, and the like), the copy number is under the control of a DNA region within the replication origin of the plasmid (ORI) which extends approximately between bases 2940 and 3130 (numbering of the bases of pBR322 proposed by Peden (Gene 22:277-280, 1983). A portion of this region, situated between bases 2970 and 3089, is transcribed into RNAs called RNAI and RNAII. RNAI, in particular, is thought to play a role in the regulation of the plasmid copy number.

The RNAII species provides an RNA primer which forms a complex at or near the origin from which DNA synthesis is initiated; the RNAI species interferes with the formation of this initiation complex (Tomizawa, Cell 47:89-97, 1986; and Lin-Chao & Cohen, Cell 65:1233-1242, 1991. Transcription of the two RNA species is controlled by separate promoter sequences associated with the DNA sequences that encode the transcripts (for reviews, see, e.g., Eguchi et.al. Biochemistry 60:631-652, 1991; and Polisky, Cell 55:929-932, 1988). In addition, there is a small polypeptide (the rop protein) that is believed to interact with the promoter for RNAII. The polypeptide is not essential for replication, however.

The origin of replication and the RNA coding sequences and their associated promoters together provide an internally self-regulated system that controls the replication incompatibility (as described below) and the copy number of these plasmids. Certain other plasmids, exemplified by RI and some Staphyloccocal plasmids, also control replication initiation at the transcriptional level, but by a messenger RNA species whose product provides an initiation factor, probably a polypeptide, which is involved in DNA replication.

Plasmids carrying a mutation that influences the copy number have been described in the art. For example, Boros et al. (Gene 30:257-260, 1984) describe a mutant plasmid derived from pBR322. The copy number of this plasmid per cell is increased by about 200-fold relative to the copy number of pBR322. The increase in the number of copies results from a G to T transversion at position 3075 on the 2846-3363 HinfI fragment, close to the 3′ end of the sequence that encodes RNAI. It had been shown previously that the same mutation in the ColE1 plasmid ColE1, which has a replication origin similar to that of pBR322, also increases the copy number of the plasmid (up to 300 per cell). See, e.g, Muesing et al., Cell, 24:235-242, 1981.

Recent advances have demonstrated the importance of regulation of the RNAI and RNAII species in controlling copy number of ColE1-derived plasmids. Several factors affect the decay of RNAI including RNase E, polynucleotide phosphorylase, poly(A) polymerase (see, e.g., Xu et.al. Proc. Natl. Acad. Sci. 90:6756-6760, 1993) and RNase III (e.g., Binnie, et.al. Microbiology 145:3089-3100, 1999).

RNase E is a single strand endonuclease that cleaves RNAI near its 5′ end and converts it to an unstable pRNAI₅, which relieves replication repression (see, e.g., Lin-Chao & Cohen, Cell 65:1233-1242, 1991). Mutations in the pcnB gene, which encodes poly(A) phosphorylase (PAP I), cause prolongation of the half-life of RNAI, and decrease the copy number of ColE1-type plasmids. PAP I adds adenosine residues to the 3′ end of RNAI, which accelerates its degradation (Xu et.al., Proc. Natl. Acad. Sci. 90:6756-6760, 1993). Alteration of the enzymatic activity of these enzymes can potentially affect copy number. Furthermore, alterations in the RNAI or RNAII species themselves may change their recognition profile for any or all of these enzymes. Further, it was also noted that the lengths of RNAI or RNAII affect their hybridization to one another (Tomizawa, Cell 47:89-97, 1986), so length of these RNAs could also be a determinant of copy number. Thus, mutations within the origin of replication that significantly alter the three dimensional conformation of RNAI or RNAII may have dramatic affects on their half-lives, interaction with one another, and ultimately plasmid copy number.

Several cloning vectors are derivatives of the ColE1-related plasmid pMB1, including pBR322 (Bolivar, et.al. Gene 2:95-113, 1997), and high-copy versions in the pUC series [e.g., Viera & Messing Gene 19:259-268, 1982; Yanisch-Perron, et.al. Gene 33:103-119, 1985) and pBluescript (Stratagene, La Jolla, Calif.). Plasmids that are compatible with pMB1 include those that use the p25A-related origins of replication (Bartolome et.al., Gene 102:75-78, 1991). In general, the copy number of these plasmids is between 15-20 copies per chromosome. While medium to low copy number vectors may be suitable for many applications, their use can be limiting when high levels of expression of a gene, or multiple genes, is required. Although replication of ColE1-like plasmids is dependent on DNA polymerase I and is regulated by the interaction of RNAI and RNAII transcripts, distinct incompatibility groups have been identified (see, e.g., Selzer, et.al. Cell 32 :119-129, 1983; and Som & Tomizawa, Mol. Gen. Genet. 187:375-383, 1982). In this regard, the segregation properties of plasmids within a cell are controlled by sequences in the origin of replication for ColE1 (Bedbrook, et.al. Nature 281:447-452, 1979). Regions of the ColE1 origin of replication critical for compatibility have been identified (see, e.g., Hashimoto-Gotoh & Inselburg, J. Bacteriol. 139:608-619, 1979). The ColE1-like plasmid RSF1030, for example, is able to reside with both pMB1 and p15A-derived plasmids, as well as with non-ColE1vectors such as pSC101. Additionally, a high copy variant of pRSF1030 with a single nucleotide change has recently been described ([Phillips, et.al. Biotechniques 28:400-408, 2000). Thus, changes within the origin of replication may alter the compatiblity phenotype of a given plasmid. However, there is a need for additional high copy number plasmids.

### SUMMARY OF THE INVENTION

The present invention provides origins of replication capable of amplifying nucleic acid at an increased copy number within a cell. In particular, the invention provides origins of replication capable of amplifying nucleic acid at an increased copy number within a prokaryotic cell, preferably a bacterium. The basis of the invention is the discovery that an insertion, a deletion, a substitution or a combination thereof in defined regions of the origin of replication result in a very high copy number and can also regulate compatibility. Preferably, the origins of replication are present on a circular polynucleotide such as a plasmid vector. Additionally, the invention provides for a cell containing one or more of said origins of replication and provides methods for producing the plasmids, genes, and gene products derived therefrom.

In one embodiment, the invention provides a plasmid that grows to a higher copy number (e.g., at least 2-fold) relative to parental plasmids. The plasmid comprises at least one mutation, e.g., an insertion, deletion, or substitution, in the origin of replication of a ColE1-type plasmid. Such mutations typically occur within the region defined as positions 1 to 210 as determined with reference to SEQ ID NO:1. In some embodiment, the mutation is within the region defined as positions 1 to 150, as determined with reference to SEQ ID NO:1. The deletion, insertion, or substitution may involve one or more positions. The deletions can be of any length, e.g., 1 to 150 base pairs, but are typically less than 100 base pairs. Similarly, the insertions may be of any length, but are typically from 1 to 100 base pairs. Substitution can occur at one ore more positions, e.g., 2, 3, 4, 5, 6, 7, 8, 9, 10, 11, 12, 15, 20, 30, 40, 50, or 100 positions. Additionally, multiple mutations and combinations of substitutions, insertions and/or deletions can also be present in an origin of replication of the invention.

Often, mutations, e.g., deletions, occur in the region of the origin encoding RNAI. Deletion mutants typically comprise deletions of varying numbers of nucleotides, e.g., from 1 to 70 nucleotides, and most often, deletions of 2, 3, 4, 5, 6, 7, 8, 9, 10, 11, 12, 13, 14, 15, 20, 25, 30, 35, 40, 45, or 50 nucleotides. Similarly, insertion mutants typically comprise insertions of varying numbers of nucleotides, e.g., from 1 to 70 nucleotides, and most often, insertions of 1, 2, 3, 4, 5, 6, 7, 8, 9, 10, 11, 12, 13, 14, 15, 20, 25, 30, 35, 40, 45, or 50 nucleotides.

Exemplary substitutions, deletions, or insertions can occur at the following positions: positions 1 to 68, positions 40 to 50, positions 57 to 60, positions 25 to 27, positions 59-64, positions 192-194, positions 128-134, positions 126-128, positions 127-129, positions 61-62, positions 93-103, positions 47-51, positions 59-65, and positions 58-63. In some embodiments, an origin of the invention comprises a sequences set forth in SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

In some embodiments, e.g., SEQ ID NO:5 or SEQ ID NO:11, a plasmid comprising an origin of replication with a mutation as described herein is compatible with other colE1-like origins and therefore can be used in a single cell with a second plasmid that comprises a different colE1 origin, e.g., a parent colE1-type origin such as that of pBluescript.

### BRIEF DESCRIPTION OF THE DRAWINGS

FIG. 1 shows a schematic of RNA regulation of ColE1-type origins of replication (thick black bar). RNAII is produced from a promotor P, and is transcribed as a sense strand. This RNA species is utilized by DNA polymerase I as a primer for DNA synthesis during initiation of replication. Control of replication is mediated by RNAI, which is transcribed in the antisense direction from a promotor P, and which mediates suppression of replication by binding to RNAII and causing a conformational change in the primer that causes inefficient extension by DNA polymerase.

FIG. 2 shows the sequence of a ColE1-related origin of replication from the pBluescript plasmid. The residues are from 1158 to 1825 of the full length plasmid. The residues encoding RNAII are in upper case and the residues encoding RNAI are underlined.

FIG. 3 shows the sequence of DNA that encodes an RNAII molecule that can prime synthesis of DNA from a ColE1-type plasmid.

FIG. 4 shows an ori5′ mutant multiple sequence alignment. The mutants are indicate by the number designation at the left. These mutations confer a high-copy number phenotype. The RNA II region is indicated in capital letters. The RNAI region is blackened. Deletions are indicated by dashes. Sequence differences in ori mutant 4.1 are indicated by underlined residues.

FIG. 5 provides exemplary data that show the change in copy number from origin of replication mutant 3.4 (right, labeled “evolved plasmid”) compared to wild-type pBluescript plasmid (left).

FIG. 6 depicts the structure of the RNAII region of pBluescript. Positions of various ColE1 deletion mutants are indicated by the ori reference number and shown as solid lines. Ori2.2 is not included in this figures, as the mutation occurs outside of the RNAII region.

### DETAILED DESCRIPTION

The present invention provides origins of replication capable of amplifying nucleic acid to an increased copy number within a cell, typically a prokaryotic cell such as a bacterium. Preferably, the origins of replication are present on a circular polynucleotide such as a plasmid vector. Additionally, the invention provides a cell containing one or more of the origins of replication of the invention. In this respect, the present invention provides origins of replication on plasmids that not only have increased copy number, but also have altered compatibilities with other plasmids. The invention also provides methods for producing the plasmids, genes, and gene products derived therefrom.

Definitions

The terms “origin” or “origin of replication” as used herein refer to a sequence of nucleic acid that will allow its replication within a cell, or in a cell free extract containing nucleic acid polymerase.

The term “ColE1-type”, “ColE1-related”, or “ColE1-derived” origin of replication refers to a member of a family of related origins of replication that have control features similar to ColE1. ColE1-related origins as defined herein are at least 70% identical, often 80% identical and typically 90% identical to SEQ ID NO:1. “ColE1-type” plasmids encode an RNAII primer that is used by DNA polymerase to initiate replication, and an RNAI molecule that regulates initiation through antisense interaction on RNAII. Most often ColE1-type plasmids replicate with a theta-type mechanism. Examples of ColE1-related origins are well known in the art and include, for example, the origins of replication of plasmids pMB 1, pBR322, the pUC series, p15A and RSF1030 (see, e.g., Selzer et.al., Cell 32:119-129, 1983). “ColE1-related” origins may be compatible or incompatible with one another.

A “high copy number plasmid” as used herein refers to a plasmid that comprises an origin of replication that results in an increase in plasmid copy number of at least 2-fold, often, 5- or 10-fold, in comparison to a control plasmid comprising the origin of replication set forth in SEQ ID NO:1

The term “compatible” as applied to plasmids refers to two or more plasmids that can exist stably together in a single cell for multiple generations. “Incompatible” plasmids are unable to be maintained stably together in a single cell for multiple generations.

The term “nucleoside” refers to a molecule comprising the covalent linkage of a pyrimidine or purine to a pentose ring (such as ribose or deoxyribose).

The term “nucleotide” refers to the phosphate ester of a nucleoside.

The term “nucleic acid” is used interchangeably with “polynucleotide” to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses complementary sequences, as well as the sequence explicitly indicated.

The term “position” as it relates to a nucleic acid sequence refers to the location of a given residue in the polynucleotide chain, not to the number of residues in a sequence per se. For example, “position” in a polynucleotide sequence is defined as the location of a nucleotide in the polynucleotide chain with reference to at least one other nucleotide. The phrase “determined with reference to” in the context of identifying changes in a nucleic acid sequence means that the nucleotide at a particular position of the reference sequence is deleted or inserted. For example, in SEQ ID NO:3, the first ten nucleotides of the sequence are GGTTTGTTTG. As determined with reference to SEQ ID NO:1, these nucleotides are at positions 69-78. Thus, the origin of replication set forth in SEQ ID NO:3 has a deletion of nucleotides 1-68, relative to SEQ ID NO: 1.

The term “nucleotide deletion” as applied to a polynucleotide means that a polynucleotide has had one or more specific residues removed from one or more positions in the polynucleotide chain when the resulting polynucleotide is compared to the parental or other reference sequence.

The term “nucleotide insertion” or “nucleotide addition” means that a polynucleotide has had specific residues added to the polynucleotide chain, such that at least one of the original residues now occupies a new position in the polynucleotide when compared to the parental or other reference sequence.

The term “nucleotide substitution” as applied to a polynucleotide means that a nucleotide at a position of a nucleic acid sequence has been substituted when compared to the parental or other reference sequence.

A “subsequence” used with respect to a nucleic acid sequence refers to a segment of the nucleic acid sequence that is less than the full-length nucleic acid sequence.

The term “DNA” refers to deoxyribonucleic acid. It will be understood by those of skill in the art that where manipulations are described herein that relate to DNA they will also apply to RNA.

The term “circular DNA” as used herein refers to a nucleic acid in which no double-stranded DNA ends are present. A circular DNA may be single-stranded or double-stranded and further may, comprise single-stranded DNA ends. For example, a circular DNA will be present if single-stranded DNA ends exist but hydrogen bonding keeps the two strands of the double-stranded molecule hybridized to one another such that a double-stranded DNA end is not created by the presence of two single-stranded ends in proximity to one another. Such a circular double-stranded polynucleotide is often referred to as “nicked”. Examples of circular DNA molecules include plasmids and phagemids.

The term “random” or “random position” as applied to a polynucleotide refers to a process by which any of the specific residue positions may be selected. Random as used herein does not mean that all points or point of cleavage or nucleotides or positions are selected or chosen with equal frequency. Rather random focuses on the unpredictable nature of the process, i.e. the worker cannot predict a priori where an event will occur or what position any base will have. Finally, not all positions need be available for cleavage for the process to be random as to the available positions or bases. For example, a polynucleotide with a length of N may have any or all of its positions (i.e. 1, 2, . . . N) affected by a manipulation. In the addition (insertion) or deletion of residues, a polynucleotide necessarily must have covalent bonds (such as phosphodiester bonds) cleaved, thereafter which residues are deleted or added (i.e. the total number of positions is decreased or increased, respectively). In describing “deletions at random positions” in a polynucleotide of length N, it is meant that any or all of the N (in a circular polynucleotide) or N−1 (in a linear polynucleotide) covalent linkages between nucleotides (i.e. phosphodiester bonds) are broken, and at least one nucleotide at the end is removed prior to re-ligation. Thus, in a process causing “deletions at random positions” the final length of the polynucleotide (N, or the number of positions) necessarily decreases. Similarly, In describing “insertions at random positions” in a polynucleotide of length N, it is meant that any or all of the N (in a circular polynucleotide) or N−1 (in a linear polynucleotide) covalent linkages between nucleotides (i.e. phosphodiester bonds) are broken, and at least one new nucleotide (i.e. a new position) is added at the end prior to re-ligation. Thus, in a process causing “insertions at random positions” the final length of the polynucleotide (N, or the number of positions) necessarily increases. It is recognized that a combination of processes involving “deletions at random positions” and “insertions at random positions” may allow the final length of the polynucleotide to remain unchanged (i.e. the additions cancel out the deletions and the final number of positions remains the same, however the nucleotides occupying the positions may be different). In describing “random cleavage” or a “single random break” in a polynucleotide of length N, it is meant that any one of the N (in a circular polynucleotide) or N−1 (in a linear polynucleotide) covalent linkages between residue positions in a single polynucleotide molecule are cleaved. Accordingly, in one vessel containing many copies of a polynucleotide, a single random break can occur at different positions in different molecules.

As used herein, “substantially pure” means an object species is the predominant species present (i.e., on a molar basis it is more abundant than any other individual macromolecular species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. Solvent species, small molecules (<500 Daltons), and elemental ion species are not considered macromolecular species.

The term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later. Preferably the region of identity is greater than about 5 bp, more preferably the region of identity is greater than 10 bp. Thus, “homologs” are nucleic acid molecules that are not identical but are capable of hybridizing to one another under physiological conditions. Double-stranded homologs are capable of hybridizing to one another following denaturation.

The term “heterologous” means that one single-stranded nucleic acid sequence is unable to hybridize to another single-stranded nucleic acid sequence or its complement. Thus areas of heterology means that nucleic acid fragments or polynucleotides have areas or regions in the sequence which are unable to hybridize to another nucleic acid or polynucleotide. Such regions are, for example, regions that are mutated.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Such washes can be performed for 5, 15, 30, 60, 120, or more minutes. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The terms “identical” or percent “identity,” in the context of two or more nucleic acid sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 30, 35, 40, 45, 50, 55, 60, 65, 70, 75, 80, 85, 90, 95, 100, 105, 110, 115, 120, 125, 130, 135, 140, 145, or 150 or more in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local alignment algorithm of Smith & Waterman, Adv. Appl. Math. 2:482 (1981), by the global alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Typically, the Smith & Waterman alignment with the default parameters are used for the purposes of this invention.

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, typically with the default parameters, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “amplification” means that the number of copies of a nucleic acid sequence is increased.

The term “wild-type” means that the nucleic acid fragment does not comprise any mutations. As used herein, the term “wild type” is equivalent to “parental sequence”, i.e., a starting or reference sequence prior to the manipulation of the sequence. For example, a mutation in the origin of the ColE1 plasmid has long been known to increase the copy number of the plasmid (up to 300 per cell). See, e.g, Muesing et al., Cell, 24:235-242, 1981. This origin can be considered to be a wild-type origin in the context of this invention.

The term “chimeric polynucleotide” means that the polynucleotide comprises nucleotide regions which are wild-type and regions that are mutated. It may also mean that the polynucleotide comprises wild-type regions from one polynucleotide and wild-type regions from another related polynucleotide.

The term “population” as used herein means a collection of components such as polynucleotides, nucleic acid fragments or proteins. A “mixed population” means a collection of components which belong to the same family of nucleic acids or proteins (i.e. are related) but which differ in their sequence (i.e. are not identical) and hence in their biological activity. A “library” necessarily implies a population wherein at least two of the components is different in some aspect (chemical composition, length, etc.).

The term “specific nucleic acid fragment” means a nucleic acid fragment having certain end points and having a certain nucleic acid sequence. Two nucleic acid fragments wherein one nucleic acid fragment has the identical sequence as a portion of the second nucleic acid fragment but different ends comprise two different specific nucleic acid fragments. Two nucleic acid fragments with identical sequences but different 5′ or 3′ ends comprise two different specific nucleic acid fragments.

The term “mutations” as used herein refers to changes in the sequence of a parental nucleic acid sequence. Mutations may be point mutations such as transitions or transversions, or deletion or insertions.

In the polynucleotide notation used herein, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the coding RNA transcript are referred to as “downstream sequences”.

As used herein the term “physiological conditions” refers to temperature, pH, ionic strength, viscosity, and like biochemical parameters which are compatible with a viable organism, and/or which typically exist intracellularly in a viable cultured yeast cell or mammalian cell. For example, the intracellular conditions in a yeast cell grown under typical laboratory culture conditions are physiological conditions. Suitable in vitro reaction conditions for in vitro transcription cocktails are generally physiological conditions. In general, in vitro physiological conditions comprise 50-200 mM NaCl or KCl, pH 6.5-8.5, 20-45° C. and 0.001-10 mM divalent cation (e.g., Mg⁺⁺, Ca⁺⁺); preferably about 150 mM NaCl or KCI, pH 7.2-7.6, 5 mM divalent cation, and often include 0.01-1.0 percent nonspecific protein (e.g., BSA). A non-ionic detergent (Tween, NP-40, Triton X-100) can often be present, usually at about 0.001 to 2%, typically 0.05-0.2% (v/v). Particular aqueous conditions may be selected by the practitioner according to conventional methods. For general guidance, the following buffered aqueous conditions may be applicable: 10-250 mM NaCl, 5-50 mM Tris HCl, pH 5-8, with optional addition of divalent cation(s) and/or metal chelators and/or nonionic detergents and/or membrane fractions and/or antifoam agents and/or scintillants.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

Introduction

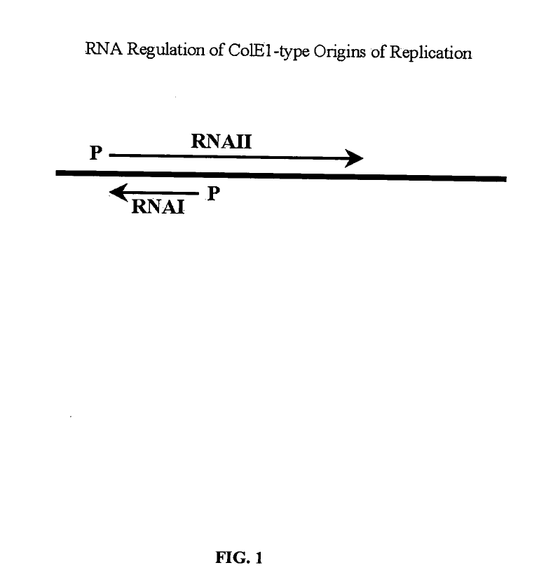

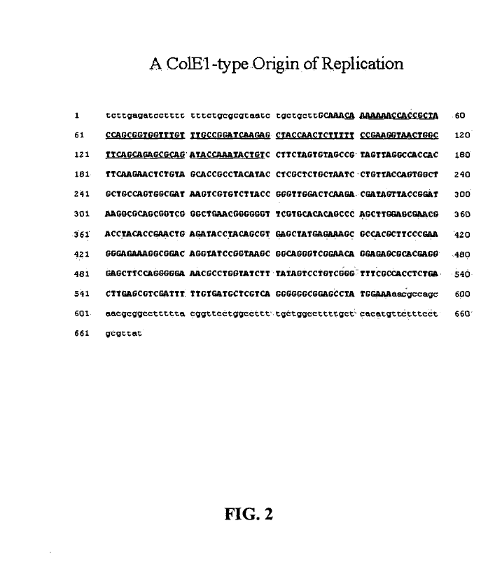

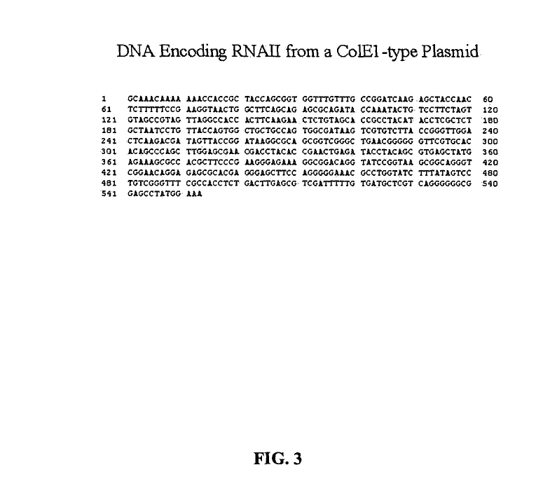

Copy number is a genetic characteristic of each plasmid. For example, in the ColE1-type plasmids (such as plasmids of the families pBR, pUC, and the like), the copy number is under the control of a DNA region corresponding to the replication origin of the plasmid (ORI) [Peden, et.al. Gene, 22 (1983) 277-280]). A portion of this region, situated between bases 2970 and 3089 is transcribed into RNAs called RNAI and RNAII. RNAI, in particular, is known to play a role in the regulation of the plasmid copy number. A schematic of a ColE1-related origin of replication is shown if FIG. 1. FIG. 2 shows the sequence of a ColE1-related origin of replication from the pBluescript plasmid. FIG. 3 shows the DNA sequence encoding RNAII from a ColE1-type plasmid. The present invention provides for mutations, often insertions or deletions, in the RNAI region of ColE1-related origins of replication that increase the copy number of plasmids that harbor them, or alter the compatibility of such plasmids.

Despite the recent advances in understanding the regulation of ColE1-type plasmids by RNAI, RNAII and specific enzyme activities, relatively few gain of function alterations in the origin of replication are known that stably increase the copy number. Boros et al. [Gene, 30, (1984) 257-260] describe a mutant plasmid derived from pBR322. The copy number of this plasmid per cell is increased by about 200-fold relative to the copy number of pBR322. This increase in the number of copies results from a G to T transversion localized in position 3075 on the 2846-3363 HinfI fragment, close to the 3′ end of the sequence transcribed into RNAI. Muesing et al. [Cell, 24 (1981) 235-242] had earlier demonstrated the same mutation in the plasmid ColE1(whose replication origin is similar through the sequence to that of pBR322), also with an increase in the copy number of the said plasmid (up to 300 per cell). Also, a high copy variant of pRSF1030 with a single nucleotide change in the region encoding RNAI has recently been described [Phillips, et al. Biotechniques 28 (2000) 400-408]. Thus, a few base pair changes have been described which increase the copy number of low to medium copy number plasmids, however increases in copy number at a magnitude of 3 fold or greater due to an insertion or deletion or alterations in compatibility have not been demonstrated to date.

Replication Origins

For a review of plasmid origin of replication families, see, e.g., del Solar, et.al. in Microbiology and Molecular Biology Reviews, 62 (1998) 434-464. Origins of replication include ColE1 family origins as well as others that are distinct from ColE1. Those that do not belong to the ColE1-related family include those derived from the plasmids R1, R6K, pSC101, or pPS10.

ColE1-type origins of replication are common in plasmids frequently used in recombinant techniques. Examples include the pBR origin and the ColE1-type origin of replication sequence comprising residues 1158 to 1825 of pBluescript. These residues are set forth in FIG. 2 and SEQ ID NO:1.

Mutations, e.g., substitutions, deletions, and/or insertions may be introduced into the origin or replication using a number of methods. Current methods in widespread use for creating mutant sequences, for example in a library format, are error-prone polymerase chain reaction (Caldwell & Joyce, (1992); Gram et al., Proc Natl Acad Sci 89:3576-80, 1992) and cassette mutagenesis Arkin & Youvan, Proc Natl Acad Sci 89:7811-5, 1992; Hermes et al., Proc Natl Acad Sci 87:696-700, 1990; Oliphant et al., Gene 44: 177-83 (1986); Stemmer & Morris, Biotechniques 13: 214-20 (1992)], in which the specific region to be optimized is replaced with a synthetically mutagenized oligonucleotide. Alternatively, mutator strains of host cells have been employed to add mutational frequency (Greener et al., Mol Biotechnol 7:189-95, 1997). In each case, a ‘mutant cloud’ Kauffman, (199) is generated around certain sites in the original sequence.

Methods of saturation mutagenesis utilizing random or partially degenerate primers that incorporate restriction sites have also been described (Hill et al., Methods Enzymol 155:558-68; 1987; Oliphant et al., Gene 44:177-83; 1986; Reidhaar-Olson et al., Methods Enzymol 208:564-86, 1991). A protocol has also been developed by which synthesis of an oligonucleotide is “doped” with non-native phosphoramidites, resulting in randomization of the gene section targeted for random mutagenesis Wang & Hoover, J Bacteriol 179:5812-9, 1997). This method allows control of position selection, while retaining a random substitution rate. Zaccolo & Gherardi, (J Mol Biol 285:775-83, 1999) describe a method of random mutagenesis utilizing pyrimidine and purine nucleoside analogs. U.S. Pat. No. 5,798,208 describes a “walk through” method, wherein a predetermined amino acid is introduced into a targeted sequence at pre-selected positions.

Methods for mutating a target gene by insertion and/or deletion mutations have also been developed. It has been demonstrated that insertion mutations could be accommodated in the interior of staphylococcal nuclease Keefe et al., Protein Sci 3:391-401, 1994). Examples of deletional mutagenesis methods developed include the utilization of an exonuclease (such as exonuclease III or Bal31) or through oligonucleotide directed deletions incorporating point deletions Ner et al., Nucleic Acids Res 17:4015-23, 1989). Additionally, Lietz describes a method whereby oligonucleotides with random sequences may be combined with PCR to induce insertions and deletions. Enhancement of function by this technique has not been shown, and the capacity to overmutagenize (i.e., make too many insertions or deletions per polynucleotide) is substantial in this method (see, e.g., U.S. Pat. No. 6,251,604.

A technique often used to evolve proteins in vitro is known as “DNA Shuffling”. In this method, a library of gene modifications is created by fragmenting homologous sequences of a gene, allowing the fragments to randomly anneal to one another, and filling in the overhangs with polymerase. A full length gene library is then reconstructed with polymerase chain reaction (PCR). The utility of this method occurs at the step of annealing, whereby homologous sequences may anneal to one another, producing sequences with attributes of both starting sequences. In effect, the method affects recombination between two or more genes that are homologous, but that contain significant differences at several positions. It has been shown that creation of the library using several homologous sequences allows a sampling of more sequence space than using a randomly mutated single starting sequence (see, e.g., Crameri et al., Nature 391:288-91, 1998). This effect is likely due to the fact that years of evolution have already selected for different advantageous or neutral mutations amongst the homologs of the different species. Starting with homologs, then, appreciably limits the number of deleterious mutations in the creation of the library which is to be screened. Combinatorially rearranging the advantageous positions of the homologs can apparently allow for an optimized secondary protein structure for catalyzing a biochemical reaction. The resulting evolved protein appears to contain positive features contributed from each of the starting sequences, which results in drastically improved function following selection.

A recently described technology describes the ability to make deletions or additions to random positions within a circular polynucleotide (see, e.g, WO0216642). This technology is especially suited to the application of producing high-copy variants of ColE1-type plasmids due to the known importance of RNA secondary structure in replication initiation. Insertions or deletions of varying length can be made at any position in the origin of replication, and a screen for high copy-number can be done to identify useful mutants.

Position of Mutations in the Origin of Replication

Origins of replication of the invention that allow circular DNA molecules to replicate to a high copy number or that confer compatibility on a plasmid can be generated by mutating, e.g., inserting, substituting, or deleting, residues at a position between, and including, position 1 to 210, as determined with reference to SEQ ID NO:1. Often, high copy number and/or compatibility origins of the invention comprise mutations, relative to SEQ ID NO:1, within the region corresponding to SEQ ID NO:1 from position 1 to 150. For example, origins of replication of the invention can have deletions of one or more nucleotides at a position in the region of SEQ ID NO:1 from position 1 to 150. Such deletions can occur at any position. Additionally, deletions can be at more than one position. The deletions vary in length. Deletions are usually less than 100 residues, often less than 50 residues, and most often less than 25, 20, or 15 residues, e.g., 12, 11, 10, 9, 8, 7, 6, 5, 4, 3, 2, or 1 residues.

Similarly, insertions in origins can occur at any of positions 1 to 210 of SEQ ID NO:1. Typically, insertions are in positions 1 to 150 of SEQ ID NO:1. Such insertions can be at any position. Further, multiple insertions can be present in the origins of the invention. The insertions vary in length. For example, the insertions are usually less than 100 base pairs, often less than 50 base pairs, and most often less than 25, 20, or 15 residues, e.g., 12, 11, 10, 9, 8, 7, 6, 5, 4, 3, 2, or 1.

Substitutions may also be introduced into the region comprising positions 1 to 210, of SEQ ID NO:1, typically positions 1 to 150 of SEQ ID NO:1. Typically, more than one position is substituted. For example, usually less than 100 positions are substituted, often less than 50 positions, and most often less than 25, 20, or 15 positions, e.g., 12, 11, 10, 9, 8, 7, 6, 5, 4, 3, 2, or 1 position.

Additionally, combinations of substitutions, insertions, or deletions as described above can be present in a ColE1-type plasmid of the invention. For example, an origin of the invention can comprise both a deletion and substitutions at other positions relative to SEQ ID NO:1, e.g, ori mutant 4.1.

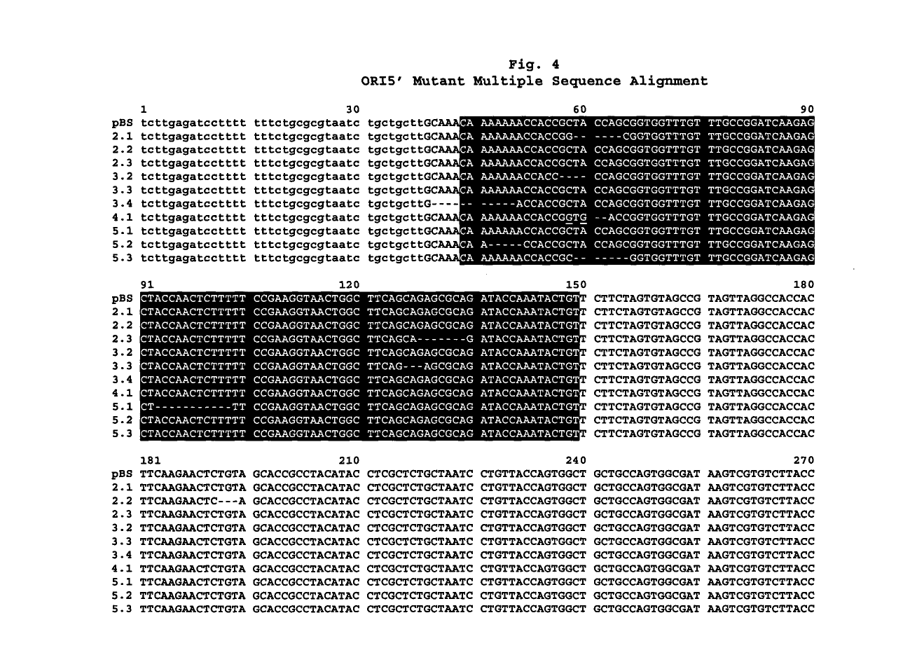

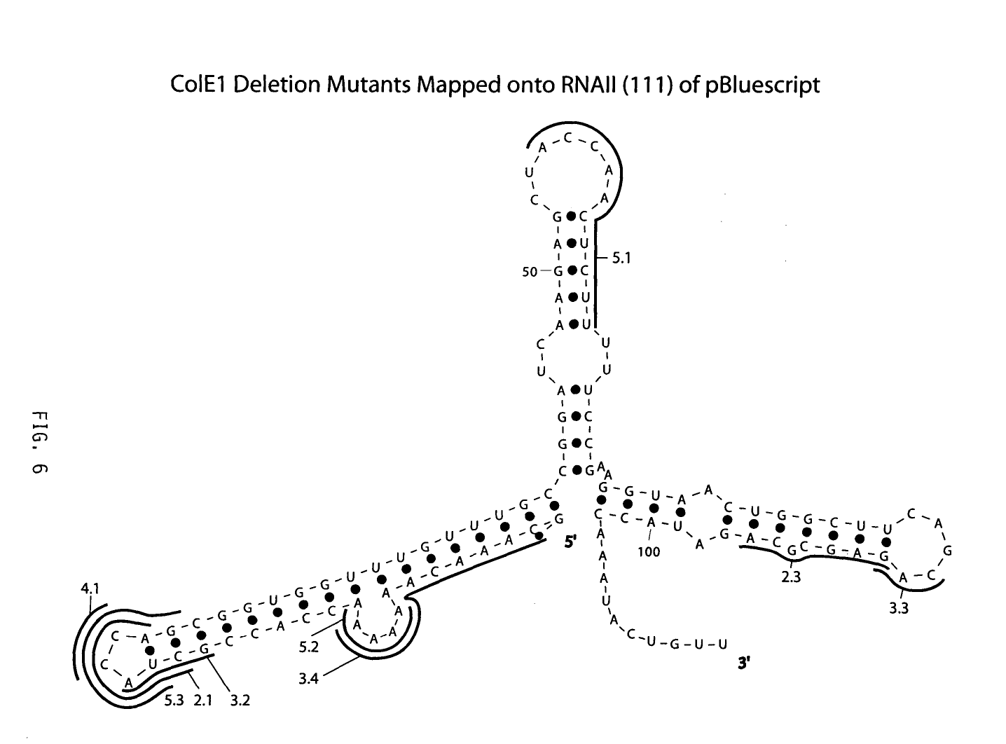

In preferred embodiments, the mutations in the origin occur in the region encoding RNAI (identified in FIGS. 2 and 4), or within 10 nucleotides of the region encoding RNAI. Often, mutations occur within the region from position 39 to position 66 of SEQ ID NO:1, or positions 91 through 135 of SEQ ID NO:1. FIG. 6 shows the structure of the RNA II region of Bluescript that includes the RNAI sequences. The positions of the ori mutations described below are shown on the structure, except for ori 2.2, where the deletion occurs outside of the RNAI region.

The invention includes, but is not limited to, the following embodiments. The positions of the residues are indicated with reference to SEQ ID NO:1.

(a) Origin mutant 3.1 (SEQ ID NO:3) comprises a deletion of 168 residues, 68 of which are at the 5′ end of the origin of replication and include the 5′ 30 positions of the DNA encoding RNAII. The other 100 residues of the deletion are outside of the reference origin as shown in SEQ ID NO:1.

(b) Origin mutant 3.2 (SEQ ID NO:4) comprises a 4 nucleotide GCTA deletion from positions 57 to 60 in the origin of replication, which corresponds to positions 18 to 21 of the RNAII transcript.

(c) Origin mutant 3.3 (SEQ ID NO:5) comprises a 3 nucleotide GCA deletion from position 125 to 127 of the origin, which corresponds to position 86 to 88 of RNAII. This can also be considered to be a 3 nucleotide deletion of CAG at positions 126 to 128, as this results in the same sequence in ori 3.3.

(d) Origin mutant 3.4 (SEQ ID NO:6) comprises an 11 nucleotide CAAACAAAAAA deletion from positions 40 to 50 of the origin, which corresponds to positions 2 to 12 of RNAII.

(e) Origin mutant 2.1 (SEQ ID NO:7) has a 6 base deletion from nucleotides 59-64 (relative to pBluescript.

(f) Origin mutant 2.2 (SEQ ID NO:8) has a 3 base deletion from 192-194.

(g) Origin mutant 2.3 (SEQ ID NO:9) has a 7 base deletion from 128-134.

(h) Origin mutant 4.1 (SEQ ID NO:10) has a two base deletion at 61-62, but also has two nucleotides altered near the deletion site (C58G and A60G).

(i) Origin mutant 5.1 (SEQ ID NO:11) has an 11 base deletion from 93-103.

(j) Origin mutant 5.2 (SEQ ID NO:12) has a 5 base deletion from 47-51.

(k) Origin mutant 5.3 (SEQ ID NO:13) has a 7 base deletion from 59-65.

Alignments of the ori 5′ regions of SEQ ID NOs:4-13 with the corresponding region of pBluescript are shown in FIG. 4.

Methods to prepare the polynucleotides comprising high-copy number origins of replication of the present invention are well known in the art. Plasmids containing expected high-copy number origins of replication can be readily assayed as described below to determine the particular plasmid's copy number. The production of high-copy plasmids could be accomplished through various mutagenesis process, e.g., site-directed mutagenesis. See, for example, Sambrook & Russell, Molecular Cloning: A Laboratory Manual, Cold Spring Harbor, 2001, 3rd edition “Oligonucleotide-Mediated Mutagenesis,” which is incorporated herein by reference. Site-directed mutagenesis is generally accomplished by site-specific primer-directed mutagenesis. This technique is now standard in the art and is conducted using a synthetic oligonucleotide primer complementary to a single-stranded phage DNA to be mutagenized except for a limited deletion or insertion representing the desired mutation. Briefly, the synthetic oligonucleotide is used as a primer to direct synthesis of a strand complementary to the plasmid or phage, and the resulting double-stranded DNA is transformed into a phage-supporting host bacterium. The resulting bacteria can be assayed by, for example, DNA sequence analysis or probe hybridization to identify those plaques carrying the desired mutated gene sequence. Alternatively, “recombinant PCR” methods can be employed [Innis et al. editors, PCR Protocols, San Diego, Academic Press, 1990, Chapter 22, Entitled “Recombinant PCR”, Higuchi, pages 177-183].

Use of the present invention typically would involve the construction of a circular polynucleotide comprising a high-copy origin of replication as described herein. Techniques for the construction of such recombinant DNA molecules are well known in the art, as described, for example, in Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York (1997). Such a high-copy plasmid may also comprise a gene of interest, which is to be expressed in a host cell. The gene of interest may be determined by the individual interest of the investigator. Such a gene may encode a pharmaceutical, an industrial enzyme, or any other RNA or protein molecule. The high-copy plasmid is preferably inserted into a host cell, preferably a prokaryote. Methods for inserting genes into prokaryotes include electroporation, heat shock transformation, and phage transduction.

The host strains suitable for the multiplication of the plasmids conforming to the invention and to the expression of the genes carried by these plasmids are the same as those which permit the multiplication of the corresponding wild type plasmids and the expression of the genes which they carry, and the behaviour and the growth of the strains transformed by the plasmids conforming to the invention are identical to those of the strains carrying the wild type plasmids.

The subject of the present invention is in addition a process for the multiplication of the plasmids conforming to the invention, which process is characterized in that, in a first step, an appropriate host bacterial strain is transformed with at least one of the said plasmids, and in a second step, the said bacterial strain is cultured.

The invention also encompasses:

A process for the amplification of a DNA sequence, which process is characterized in that, in a first step, the said sequence is inserted in a plasmid conforming to the invention, and in that, in a second step, the multiplication of the said plasmid is carried out as indicated above.

A process for the production of polypeptides by genetic engineering, which process is characterized in that, in a first step, the gene encoding the said polypeptide is inserted in a plasmid conforming to the invention, in a second step, an appropriate host bacterial strain is transformed with the said plasmid, and in a third step, the said bacterial strain is cultured under conditions appropriate for the expression of the said gene.

Determination of Plasmid Copy Number

Relative Copy Number

One method to determine the relative copy number of plasmids is described in U.S. Pat. No. 4,703,012. In this method, plasmids of a normal copy number are cultured in parallel with a test high-copy plasmid. The bacteria are lysed, and the plasmids are compared by agarose gel electrophoresis at various dilutions. If the test plasmid stains more intensely with ethidium bromide at a given dilution compared to the normal copy plasmid, then its copy number is increased by an amount proportional to the increase in staining. The plasmid of normal copy number may be any ColE1-related plasmid. Examples of such ColE1-related plasmids are pMB1, pBR322, the pUC series, p15A, and the pBluescript series. A test plasmid may be any ColE1-related plasmid that is suspected of having a high copy number.

Alternatively, plasmid copy number can be determined as a proportion of chromosome copies. In this method, bacterial cells are lysed, protein is digested by a protease, and total DNA is analyzed by agarose gel electrophoresis. The relative amounts of plasmid to chromosomal DNA can be determined for a normal copy plasmid, and compared to a test high-copy plasmid. This comparison can be quantified using ethidium bromide staining of said agarose gels. Normal copy plasmids could be plasmids harboring ColE1-related origins of replication such as pMB1, pBR322, the pUC series, p15A, and the pBluescript series.

Absolute Copy Number

The absolute copy number of a plasmid within a cell can be determined by analyzing the average number of plasmid molecules within a cell in a given culture. In this method, a culture of cells is grown containing the test plasmid, and an aliquot of cells are lysed in mid log phase. Plasmid DNA is prepared from this aliquot by any of several standard techniques. The plasmid DNA concentration, and absolute amount, are determined by spectroscopy or fluorometry. The remaining cells are then plated in multiple dilutions on LB plates with the appropriate antibiotic selection. The colonies growing on these plates are then counted to give an accurate measure of the viable cells in the original culture. The copy number is then determined by deducing the number of copies/viable cell using the data acquired in the aforementioned process. Alternatively, the optical density of a bacterial colony often relates linearly to its cell count. Hence, optical density can be used as the denominator of the preceding calculation.

Plasmid Compatibility Testing

Compatibility of plasmids may be tested by inserting two or more plasmids into the same bacterial cell. The plasmids should preferably have some distinguishing characteristic between them. The distinguishing characteristic may be resistance to an antibiotic, as is well known in the art. For example, one plasmid may confer resistance to ampicillin by harboring a beta-lactamase gene, whereas the second plasmid may confer resistance to a different antibiotic, such as tetracycline or chloramphenicol. When the bacterial cells are selected in the presence of both antibiotics, all of the cells in that population will harbor both plasmids. When one of the antibiotics is removed, cells with compatible plasmids will retain the second plasmid, whereas incompatible plasmids will lose the plasmid that is not under selection pressure. Cells retaining the second plasmid can be identified by replica plating the cells from plates containing the first antibiotic onto new agar plates which contain the second antibiotic by techniques well known to those skilled in the art.

### EXAMPLES

The following examples are provided by way of illustration only and not by way of limitation. Those of skill in the art will readily recognize a variety of noncritical parameters that could be changed or modified to yield essentially similar results.

Four plasmids derived from the ColE1-related pBluescript were constructed according to known methodology (see, e.g., WO0216642). These plasmids were identified based on their increased expression of beta-galactosidase, which requires the alpha peptide encoded by the plasmids.

Beta-galactosidase expression was evaluated by plating the TOP10F′ bacteria on LB agar plates containing the colorimetric substrate X-Gal, either with or without the inducer of lacZ IPTG. The four origin of replication mutants showed increased blue colony color compared to the wild-type pBluescript plasmid, both in the presence and in the absence of the inducer IPTG. The number of copies per viable cell was determined by growing 100 ml cultures of DH10B e.coli containing each of the plasmids, preparing the plasmids from a 50 ml aliquot by QIAGEN columns (QIAGEN, Chatsworth, Calif.), determining the absolute amount of plasmid DNA in the preparation by O.D.₂₆₀, and plating dilutions of the remaining 50 ml of culture in order to determine the number of viable cells in the original culture.

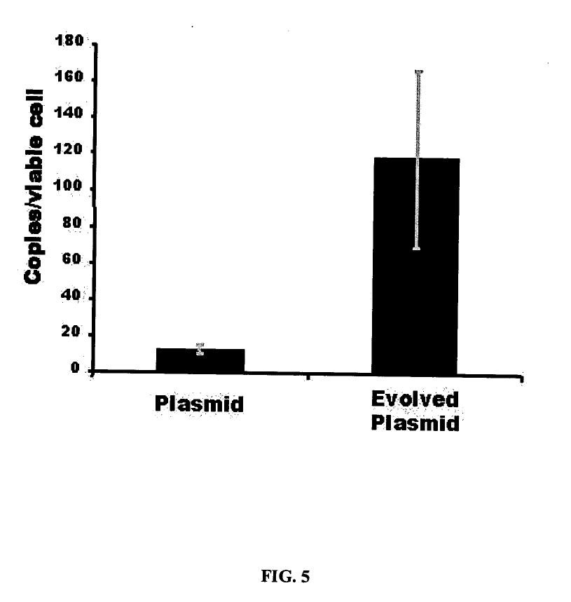

An exemplary experiment shows that the copy number per viable cell was increased nearly ten fold compared to wild-type plasmids (FIG. 5). The plasmids were sequenced and the only alterations from wild-type were in the origin of replication. The sequences of the origins of replication are set forth in SEQ ID NOs:3-6.

Additional plasmids were also constructed as above. Sequences of high copy number plasmids identified as set forth herein are shown in SEQ ID NOs:7-14.

Of these sequences, ori 3.3 and ori 5.1 are compatibility mutants, i.e., these plasmids can co-exist with ColE1-related origins, e.g., wild-type ColE1origins, in a single cell.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications and patent applications cited in this specification are herein incorporated by reference as if each individual publication or patent application were specifically and individually indicated to be incorporated by reference.

Table of Sequences

SEQ I.D NO: 1 ColE1-related
origin from pBluescript (residues 1158-1825)
TCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTC
TTTTTCCGAAGGTAACTGGCTTCAGCAGAGCGCAGATACCAAATACTGTC
CTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTG
GCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTT
GGAGCGAACGACCTACACCGAACTGAGATACCTACAGCGTGAGCTATGAG
AAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACAT
GTTCTTTCCTGCGTTAT
SEQ ID NO: 2 DNA encoding RNAII
from the ColE1-related plasmid pBluescript
GCAAACAAAAAAACC ACCGCTACCAGCGGT GGTTTGTTTGCCGGA
TCAAGAGCTACCAAC TCTTTTTCCGAAGGT AACTGGCTTCAGCAG
AGCGCAGATACCAAA TACTGTCCTTCTAGT GTAGCCGTAGTTAGG
CCACCACTTCAAGAA CTCTGTAGCACCGCC TACATACCTCGCTCT
GCTAATCCTGTTACC AGTGGCTGCTGCCAG TGGCGATAAGTCGTG
TCTTACCGGGTTGGA CTCAAGACGATAGTT ACCGGATAAGGCGCA
GCGGTCGGGCTGAAC GGGGGGTTCGTGCAC ACAGCCCAGCTTGGA
GCGAACGACCTACAC CGAACTGAGATACCT ACAGCGTGAGCTATG
AGAAAGCGCCACGCT TCCCGAAGGGAGAAA GGCGGACAGGTATCC
GGTAAGCGGCAGGGT CGGAACAGGAGAGCG CACGAGGGAGCTTCC
AGGGGGAAACGCCTG GTATCTTTATAGTCC TGTCGGGTTTCGCCA
CCTCTGACTTGAGCG TCGATTTTTGTGATG CTCGTCAGGGGGGCG
GAGCCTATGGAAA
SEQ ID NO: 3 DNA encoding origin 3.1,
a high-copy variant of a ColE1 plasmid.
GGTTTGTTTGCCGGA TCAAGAGCTACCAAC TCTTTTTCCGAAGGT
AACTGGCTTCAGCAG AGCGCAGATACCAAA TACTGTCCTTCTAGT
GTAGCCGTAGTTAGG CCACCACTTCAAGAA CTCTGTAGCACCGCC
TACATACCTCGCTCT GCTAATCCTGTTACC AGTGGCTGCTGCCAG
TGGCGATAAGTCGTG TCTTACCGGGTTGGA CTCAAGACGATAGTT
ACCGGATAAGGCGCA GCGGTCGGGCTGAAC GGGGGGTTCGTGCAC
ACAGCCCAGCTTGGA GCGAACGACCTACAC CGAACTGAGATACCT
ACAGCGTGAGCTATG AGAAAGCGCCACGCT TCCCGAAGGGAGAAA
GGCGGACAGGTATCC GGTAAGCGGCAGGGT CGGAACAGGAGAGCG
CACGAGGGAGCTTCC AGGGGGAAACGCCTG GTATCTTTATAGTCC
TGTCGGGTTTCGCCA CCTCTGACTTGAGCG TCGATTTTTGTGATG
CTCGTCAGGGGGGCG GAGCCTATGGAAAAA CGCCAGCAACGCGGC
CTTTTTACGGTTCCT GGCCTTTTGCTGGCC TTTTGCTCACATGTT
CTTTCCTGCGTTAT
SEQ ID NO: 4 DNA encoding origin 3.2,
a high-copy variant of a ColE1 plasmid.
TCTTGAGATCCTTTTTTTCTGCGCGTAATCTGCTGCTTGCAAACAAAAAA
ACCACCCCAGCGGTGGTTTGTTTGCCGGATCAAGAGCTACCAACTCTTTT
TCCGAAGGTAACTGGCTTCAGCAGAGCGCAGATACCAAATACTGTCCTTC
TAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGCGGTCGGGCTGAACGGGGGGTTCGTGCACACAGCCCAGCTTGGAG
CGAACGACCTACACCGAACTGAGATACCTACAGCGTGAGCTATGAGAAAG
CGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACG
CGGCCTTTTTACGGTTCCTGGCCTTTTGCTGGCCTTTTGCTCACATGTTC
TTTCCTGCGTTAT
SEQ ID NO: 5 DNA encoding origin 3.3,
a high-copy variant of a ColE1 plasmid.
TCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTC
TTTTTCCGAAGGTAACTGGCTTCAGAGCGCAGATACCAAATACTGTCCTT
CTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCG
ATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGA
GCGAACGACCTACACCGAACTGAGATACCTACAGCGTGAGCTATGAGAAA
GCGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGAT
TTTTGTGATGCTCGTCAGGGGGGCGGAGCCTATGGAAAAACGCCAGCAAC
GCGGCCTTTTTACGGTTCCTGGCCTTTTGCTGGCCTTTTGCTCACATGTT
CTTTCCTGCGTTAT
SEQ ID NO: 6 DNA encoding origin 3.4,
a high-copy variant of a ColE1 plasmid
TCTTGAGATCCTTTTTTTCTGCGCGTAATCTGCTGCTTGACCACCGCTAC
CAGCGGTGGTTTGTTTGCCGGATCAAGAGCTACCAACTCTTTTTCCGAAG
GTAACTGGCTTCAGCAGAGCGCAGATACCAAATACTGTCCTTCTAGTGTA
GCCGTAGTTAGGCCACCACTTCAAGAACTCTGTAGCACCGCCTACATACC
TCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCG
GTCGGGCTGAACGGGGGGTTCGTGCACACAGCCCAGCTTGGAGCGAACGA
CCTACACCGAACTGAGATACCTACAGCGTGAGCTATGAGAAAGCGCCACG
CTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTT
ATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTT
TTTACGGTTCCTGGCCTTTTGCTGGCCTTTTGCTCACATGTTCTTTCCTG
CGTTAT
SEQ ID NO: 7 DNA encoding ori 2.1,
a high-copy variant of a ColE1 plasmid
TCTTGAGATCCTTTTTTTCTGCGCGTAATCTGCTGCTTGCAAACAAAAAA
ACCACCGGCGGTGGTTTGTTTGCCGGATCAAGAGCTACCAACTCTTTTTC
CGAAGGTAACTGGCTTCAGCAGAGCGCAGATACCAAATACTGTTCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTAT
SEQ ID NO: 8 DNA encoding ori 2.2,
a high-copy variant of a ColE1 plasmid
TCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTC
TTTTTCCGAAGGTAACTGGCTTCAGCAGAGCGCAGATACCAAATACTGTT
CTTCTAGTGTAGCCGTAGTTAGGCCACCACTTCAAGAACTCAGCACCGCC
TACATACCTCGCTCTGCTAATCCTGTTACCAGTGGCTGCTGCCAGTGGCG
ATAAGTCGTGTCTTACCGGGTTGGACTCAAGACGATAGTTACCGGATAAG
GCGCAGCGGTCGGGCTGAACGGGGGGTTCGTGCACACAGCCCAGCTTGGA
GCGAACGACCTACACCGAACTGAGATACCTACAGCGTGAGCTATGAGAAA
GCGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGAT
TTTTGTGATGCTCGTCAGGGGGGCGGAGCCTATGGAAAAACGCCAGCAAC
GCGGCCTTTTTACGGNTCCTGGNCNTTTGCTGGCCTTTTGCTCACATGTT
CTTTCCTGCGTTAT
SEQ ID NO: 9 DNA encoding ori 2.3,
a high-copy variant of a ColE1 plasmid
TCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTC
TTTTTCCGAAGGTAACTGGCTTCAGCAGATACCAAATACTGTTCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGCCTTTTGCTGGCCTTTTGCTCACATGTTCTTT
CCTGCGTTAT
SEQ ID NO: 10 DNA encoding ori 4.1,
a high-copy variant of a ColE1 plasmid
TCTTGAGATCCTTTTTTTCTGCGCGTAATCTGCTGCTTGCAAACAAAAAA
ACCACCGGTGACCGGTGGTTTGTTTGCCGGATCAAGAGCTACCAACTCTT
TTTCCGAAGGTAACTGGCTTCAGCAGAGCGCAGATACCAAATACTGTTCT
TCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGC
GATAAGTCGTGTCTTACCGGGTTGGACTCAAGACGATAGTTACCGGATAA
GGCGCAGCGGTCGGGCTGAACGGGGGGTTCGTGCACACAGCCCAGCTTGG
AGCGAACGACCTACACCGAACTGAGATACCTACAGCGTGAGCTATGAGAA
AGCGCCACGCTTCCCGAAGGGAGAAAGGCGGACAGGTATCCGGTAAGCGG
CANGGTCGGAACAGGAGAGCGCACGANGGAGCTTCCAGGGGGAAACGCCT
GGTATCTTTATAGTCCTGTCGGGTTTCGCCACCTCTGACTTGAGCGTCGA
TTTTTGTGATGCTCGTCAGGGGGGCGGAGCCTATGGAAAAACGCCAGCAA
CGCGGCCTTTTTACGGTTCCTGGCCTTTTGCTGGCCTTTTGCTCACATGT
TCTTTCCTGCGTTAT
SEQ ID NO: 11 DNA encoding ori 5.1,
a high-copy variant of a ColE1 plasmid
TCTTGAGATCCTTTTTTTCTGCGCGTAATCTGCTGCTTGCAAACAAAAAA
ACCACCGCTACCAGCGGTGGTTTGTTTGCCGGATCAAGAGCTTTCCGAAG
GTAACTGGCTTCAGCAGAGCGCAGATACCAAATACTGTTCTTCTAGTGTA
GCCGTAGTTAGGCCACCACTTCAAGAACTCTGTAGCACCGCCTACATACC
TCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCG
GTCGGGCTGAACGGGGGGTTCGTGCACACAGCCCAGCTTGGAGCGAACGA
CCTACACCGAACTGAGATACCTACAGCGTGAGCTATGAGAAAGCGCCACG
CTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTT
ATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTT
TTTACGGTTCCTGGCCTTTTGCTGGNCTTTTNGCTCACATGTTCTTTCCT
GCGTTAT
SEQ ID NO: 12 DNA encoding ori 5.2,
a high-copy variant of a ColE1 plasmid
TCTTGAGATCCTTTTTTTCTGCGCGTAATCTGCTGCTTGCAAACAACCAC
CGCTACCAGCGGTGGTTTGTTTGCCGGATCAAGAGCTACCAACTCTTTTT
CCGAAGGTAACTGGCTTCAGCAGAGCGCAGATACCAAATACTGTTCTTCT
AGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGAT
AAGTCGTGTCTTACCGGGTTGGACTCAAGACGATAGTTACCGGATAAGGC
GCAGCGGTCGGGCTGAACGGGGGGTTCGTGCACACAGCCCAGCTTGGAGC
GAACGACCTACACCGAACTGAGATACCTACAGCGTGAGCTATGAGAAAGC
GCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGT
ATCTTTATAGTCCTGTCGGGTTTCGCCACCTCTGACTTGAGCGTCGATTT
TTGTGATGCTCGTCAGGGGGGCGGAGCCTATGGAAAAACGCCAGCAACGC
GGCCTTTTTACGGTTCCTGGCCTTTTGCTGGCCTTTTGCTCACATGTTCT
TTCCTGCGTTAT
SEQ ID NO: 13 DNA encoding ori 5.3,
a high-copy variant of a ColE1 plasmid
TCTTGAGATCCTTTTTTTCTGCGCGTAATCTGCTGCTTGCAAACAAAAAA
ACCACCGCGGTGGTTTGTTTGCCGGATCAAGAGCTACCAACTCTTTTTCC
GAAGGTAACTGGCTTCAGCAGAGCGCAGATACCAAATACTGTTCTTCTAG
TGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAA
GTCGTGTCTTACCGGGTTGGACTCAAGACGATAGTTACCGGATAAGGCGC
AGCGGTCGGGCTGAACGGGGGGTTCGTGCACACAGCCCAGCTTGGAGCGA
ACGACCTACACCGAACTGAGATACCTACAGCGTGAGCTATGAGAAAGCGC
CACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTAT
CTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGG
CCTTTTTACGGTTCCTGGCCTTTTGCTGGCCTTTTGCTCACATGTTCTTT
CCTGCGTTAT

What is claimed is:

1. A ColE1 origin of replication comprising at least one mutation at one or more nucleotides from position 1 to position 210 as determined with reference to SEQ ID NO:1, wherein the mutation increases plasmid copy number of a plasmid comprising the origin by at least 2-fold in comparison to a control plasmid comprising the origin of replication set forth in SEQ ID NO:1 or confers compatibility with a second ColE1-type origin.

2. An origin of replication of claim 1, wherein the origin comprises at least one mutation at one or more nucleotides from position 1 to position 150 as determined with reference to SEQ ID NO:1.

3. An origin of replication of claim 1, wherein the mutation is a deletion.

4. An origin of replication of claim 3, wherein the deletion is 20 or fewer nucleotides in length.

5. An origin of replication of claim 1, wherein the mutation is an insertion.

6. An origin of replication of claim 5, wherein the insertion is 20 or fewer nucleotides in length.

7. An origin of replication of claim 1, wherein the mutation is a substitution.

8. An origin of replication of claim 1, wherein the mutation occurs in a region selected from the group consisting of positions 1 to 68, positions 40 to 50, positions 57 to 60, positions 25 to 27, positions 59-64, positions 192-194, positions 128-134, positions 126-128, positions 61-62, positions 93-103, positions 47-51, positions 59-65, and positions 58-63.

9. The origin of replication of claim 1, wherein the origin comprises a sequence as set forth in SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

10. A circular DNA comprising the origin of replication of claim 1.

11. A plasmid comprising an origin of replication of claim 1, wherein the plasmid copy number is increased at least 2-fold in comparison to a control plasmid comprising the origin of replication set forth in SEQ ID NO:1.

12. A plasmid comprising the origin of replication of claim 1, wherein the plasmid is compatible with a second ColE1 plasmid.

13. A cell comprising a circular DNA, wherein the circular DNA molecule comprises a ColE1-related origin of replication as set forth in claim 1.

14. The cell of claim 13, wherein the circular DNA is a plasmid.

15. The cell of claim 14, wherein the cell comprises a second plasmid.

16. The cell of claim 15, wherein the ColE1-related origin of replication comprises SEQ ID NO:5 or SEQ ID NO:11, and the second plasmid comprises a second ColE1-related origin.

17. A method of generating a plasmid at a high copy number, the method comprising:
introducing into a bacterial cell a circular DNA comprising a ColE1-type replication origin, wherein the replication origin comprises at least one mutation at one or more nucleotides from position 1 to position 210 as determined with reference to SEQ ID NO:1, and
culturing the bacterial cell.

18. The method of claim 17, wherein the origin comprises at least one mutation at one or more nucleotides form position 1 to position 150 as determined with reference to SEQ ID NO:1.

19. The method of claim 17, wherein the mutation is a deletion.

20. The method of claim 19, wherein the deletion is 20 or fewer nucleotides in length.

21. The method of claim 17, wherein the mutation is an insertion.

22. The method of claim 21, wherein the insertion is 20 or fewer nucleotide in length.

23. The method of claim 17, wherein the mutation is a substitution.

24. The method of claim 17, wherein the deletion or insertion occurs within at least one of the regions selected from the group consisting of positions 1 to 68, positions 40 to 50, positions 57 to 60, positions 25 to 27, positions 59-64, positions 192-194, positions 128-134, positions 126-128, positions 61-62, positions 93-103, positions 47-51, positions 59-65, and positions 58-63.

25. The method of claim 17, wherein the origin comprises a sequence set forth in SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

26. The method of claim 25, wherein the origin comprises SEQ ID NO:5 or SEQ ID NO:11 and the method further comprises introducing into a bacterial cell a circular DNA comprising a second ColE1-type replication origin.
